(19) United States
(12) Patent Application Publication
(10) Pub. No.: US 2025/0283067 A1
(43) Pub. Date: Sep. 11, 2025

(54) Highly efficient and simple SSPER and rrPCR approaches for the accurate site-directed mutagenesis of large plasmids

(71) Applicant: UNIVERSITY OF FLORIDA RESEARCH FOUNDATION, INCORPORATED, Gainesville, FL (US)

(72) Inventors: Julie A. MAUPIN, Gainesville, FL (US); Huiyong JIA, Gainesville, FL (US)

(21) Appl. No.: 18/863,703

(22) PCT Filed: May 10, 2023
(86) PCT No.: PCT/US2023/021638

§ 371 Date: Nov. 7, 2024

Related U.S. Application Data
(60) Provisional application No. 63/340,209, filed on May 10, 2022.

(51) Int. Cl. C12N 15/10; C12N 9/12; C12N 15/70

(52) U.S. Cl. CPC C12N 15/102; C12N 9/1252; C12N 15/70; C12Y 207/07007

(57) ABSTRACT

Presented arc two new methods, single primer extension reaction (SSPER) and reduce recycle PCR (rrPCR). These methods have a step that easily removes the oligonucleotide primer(s) after the first reaction, thus, allowing for the addition of a second reaction in chronological sequence to generate and isolate the appropriate DNA product with the site-directed mutation(s). A high efficiency of the methods is demonstrated by generating all ten site-directed mutations and six paired combinations of these mutations on four plasmid DNA templates ranging from 10 to 12 kb and 57 to 59% GC-content at a rate of 50-100%. The methods are i) highly accurate allowing for screening of plasmids by DNA sequencing, ii) streamlined to generate the mutations within a single day, iii) cost-effective in requiring only two primers and two enzymes (Dpnl and a proofreading DNA polymerase), iv) straightforward in primer design, and v) applicable for both large and small plasmids.

### GOVERNMENT SUPPORT

This invention was made with government support under Grant No. DE-FG02-05ER15650 awarded by the Department of Energy Chicago and Grant No. GM057498 awarded by the National Institutes of Health. The government has certain rights in the invention.

### BACKGROUND

Side-directed mutagenesis is a common tool used in molecular biology to study the influence of DNA sequence on downstream biological and biochemical functions [1-3]. The methods used to perform site-directed mutagenesis are varied but often include polymerase chain reaction (PCR) to generate point substitutions, deletions, and/or insertions in the DNA. Methods that accurately generate substitution mutations are desirable for protein structure-function studies as they allow for the expression of proteins with targeted alterations in amino acid residues including the presumed active site.

PCR-based methods that generate substitution mutations are available but can have several disadvantages. One of the earliest methods is overlap extension PCR in which overlapping DNA products with the mutations are generated by two PCRs that are subsequently used as template for a final overlap extension PCR [4-6]. This final PCR product can be difficult to generate and must be subsequently cloned into a plasmid vector for expression studies making this approach time-consuming and inefficient. An alternative and more straightforward PCR approach incorporates complementary primer pairs which have the substitution mutation(s) positioned at the center of each primer [7, 8]. In this PCR method, the expression plasmid serves as the DNA template and is prepared in an E. coli dam+dcm+strain. After PCR, the template is removed from the sample using DpnI, a restriction enzyme that cleaves the methylated DNA, while leaving the unmethylated PCR product intact [9]. Due to the complementary nature of the primer pair, primer-dimers often form during PCR leading to reaction failure. DNA insertions and other artifacts can also occur in the PCR products when using this type of primer design. Modifying the primers to be partially complementary can overcome these limitations, but the downstream consequences of the homologous primer ends include the need to perform in vitro recombination to avoid DNA sequence repeats which adds complexity to the method [10, 11]. Inverse PCR is also an option, with one of the primers designed with the substitution mutation(s) near the 5′ end [12]. The PCR products are phosphorylated and ligated after amplification, with DpnI used to remove the methylated template DNA prior to transformation. One limitation to this approach is that inverse PCR can be unreliable, particularly for large DNA templates. More importantly, DNA artifacts can occur at the site of self-ligation, most likely due to the asymmetric positioning of the mutation in the primer.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIGS. 1A-B show the plasmid templates, primers, and results generated by the standard PCR compared to the sequential single primer extension reaction (SSPER) methods. FIG. 1A is a restriction map of the 10.2 kb pJAM1208 and 11.1 kb pJAM503 plasmids used as templates.

FIG. 1B is the orientation of the primers on the genes targeted for mutagenesis (left) and the tabulated results of the standard PCR and SSPER methods (right). Primers p1 and p2 are designed to anneal to the his6-hvo_1016 (JAMM2) open reading frame on plasmid pJAM1208, while primers p9 and p15 are designed to anneal to the his6-hvo_0850 (PAN1) open reading frame on plasmid pJAM503.

FIGS. 2A-B show the primer design (A) and strategy (B) used to incorporate substitution mutations in large plasmids that relies upon the sequential single primer extension reaction (SSPER) method. pF and pR, single stranded DNA primers designed to be complementary with the substitution mutation in the center of each primer as indicated. Red asterisk, the nucleotide base(s) targeted for mutagenesis. Orange box, gene targeted for mutagenesis. Methylated plasmid DNA (purple) carrying the target gene was used as the initial template as indicated. Either of the two primers can be used in the first extension reaction 1. The DNA product generated by this reaction is digested with DpnI to cleave the methylated DNA template, The DNA product is then purified from the first primer (pF) by PCR clean up. The DpnI cleaved original template fragments can be recovered along with the PCR product but cannot effectively serve as the template for the following PCR, nor can these DNA fragments be transformed into E.coli. A second single primer extension reaction is then used with the complementary primer (pR) to generate the desired DNA product. The DNA product is finally enriched and purified by PCR cleanup prior to transformation into E. coli. The DNA gaps in the plasmid are sealed by E. coli.

FIGS. 3A-B show the primer design (A) and strategy (B) used for the reduce recycle PCR (rrPCR) method for site-directed mutagenesis. pF and pR, single stranded DNA primers used in the first PCR reaction (PCR-1) with methylated plasmid DNA as the template (purple) for amplification of “long primer”. The red asterisk represents the site-directed mutation(s). Orange box, gene carried on a large plasmid used as template. The original primers (pF and pR) were removed after PCR-1 by PCR clean up. In the second PCR reaction (PCR-2), the methylated plasmid DNA was reused as template with the PCR-1 product serving as the primer pair. After PCR-2, the methylated DNA template was removed by DpnI digestion. The PCR-2 product was enriched and purified by PCR cleanup prior to transformation into E. coli. The DNA gaps in the plasmid were sealed in vivo by the cell. A similar method was used to generate two SDMs in parallel with exception that both the pF and pR primers used in PCR-1 carried an SDM.

FIGS. 4A-B show the plasmid templates, primers and positive clones generated using the reduce recycle PCR (rrPCR) method. FIG. 4A is restriction maps of the 10.2 to 11.8 kb plasmids used as templates. FIG. 4B is the orientation of the primers p3 to p14 on the open reading frames targeted for mutagenesis including his6-hvo_2995 (Fdx), his6-hvo_0850 (Pan1) and his6-hvo_1018 (RecJ3) and tabulated results.

FIG. 5 is an example of sequence artifacts that occur when using the conventional PCR with complementary primers to generate substitution mutations in large plasmids. pan1, original DNA sequence of his6-hvo_0850 (pan1) open reading frame carried on plasmid pJAM503 that was targeted for the single substitution mutation of A to T as highlighted in yellow. The region targeted for annealing by the p9 and complementary p15 primers is underlined. 2-1 and 2-2, represent the DNA sequences of the two plasmids selected from independent clones for isolation and DNA sequencing. The red and grey highlighted regions indicate regions of DNA sequence that differ from the original plasmid template.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

The term “amplifying” or “amplification” a nucleic acid sequence generally refers to the production of a plurality of nucleic acid copy molecules having that sequence from a target nucleic acid wherein primers hybridize to specific sites on the target nucleic acid molecules in order to provide an initiation site for extension by a polymerase, e.g., a DNA polymerase. Amplification can be carried out by any method generally known in the art, such as but not limited to: standard PCR, real-time PCR, long PCR, hot start PCR, qPCR, Reverse Transcription PCR and Isothermal Amplification.

The terms “annealing” or “anneal” refer to the pairing of complementary sequences of single-stranded DNA or RNA by hydrogen bonds to form a double-stranded polynucleotide. Before annealing can occur, one of the strands may need to be phosphorylated by an enzyme such as kinase to allow proper hydrogen bonding to occur. The term “annealing” is often used to describe the binding of a DNA probe, or the binding of a primer to a DNA strand during a polymerase chain reaction.

The term “digestion” as used herein in reference to the enzymatic activity of a selection enzyme is used broadly to refer both to (i) enzymes that catalyze the conversion of a polynucleotide into polynucleotide precursor molecules and to (ii) enzymes capable of catalyzing the hydrolysis of at least one bond on polynucleotides so as to interfere adversely with the ability of a polynucleotide to replicate (autonomously or otherwise) or to interfere adversely with the ability of a polynucleotide to be transformed into a host cell. Restriction endonucleases are an example of an enzyme that can “digest” a polynucleotide. Typically, a restriction endonuclease that functions as a selection enzyme in a given situation will introduce multiple cleavages into the phosphodiester backbone of the template strands that are digested. Other enzymes that can “digest” polynucleotides include, but are not limited to, exonucleases and glycosylases.

The term “high-fidelity polymerase” refers to a polymerase with a low error rate and several safeguards to protect against making or propagating mistakes while replicating nucleic acids. If an incorrect nucleotide does bind in the polymerase active site, incorporation is slowed due to the sub-optimal architecture of the active site complex. This lag time increases the opportunity for the incorrect nucleotide to dissociate before polymerase progression, thereby allowing the process to start again, with a correct nucleoside triphosphate. Some high-fidelity polymerases contain a 3′-5′ exonuclease activity known as “proofreading” to excise incorrectly incorporated mononucleotides and to replace them with the correct nucleotide. Examples of high-fidelity polymerases include, but are not limited to, Q5® High-Fidelity DNA Polymerase and Phusion® High-Fidelity DNA Polymerase.

The term “mutagenic primer” refers to an oligonucleotide primer used in an amplification reaction, wherein the primer does not precisely match the target hybridization sequence. The mismatched nucleotides in the mutagenic primer are referred to as mutation sites with respect to the mutagenic primer. Thus, during the amplification reaction, the mismatched nucleotides of the primer are incorporated into the amplification product thereby resulting in the synthesis of a mutagenized DNA strand comprising the mutagenic primer that was used to prime synthesis mutagenizing the target sequence.

The terms “nucleic acid,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The term “sequence” refers to a nucleotide sequence of any length, which can be DNA or RNA; can be linear, circular or branched and can be either single-stranded or double stranded. The term “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination there between, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

The term “site-directed mutagenesis” refers to a molecular biology method that is used to make specific and intentional mutating changes to the DNA sequence of a gene and any gene products. The basic procedure requires the synthesis of a short mutagenic primer. This synthetic primer contains the desired mutation and is complementary to the template DNA around the mutation site so it can hybridize with the DNA in the gene of interest. The mutation may be a single base change (a point mutation), multiple base changes, deletion, or insertion. The single-strand primer is then extended using a DNA polymerase, which copies the rest of the gene. The gene thus copied contains the mutated site and is then introduced into a host cell in a vector and cloned. Finally, mutants are selected by DNA sequencing to check that they contain the desired mutation.

The term “template” refers to the molecule that acts as a pattern for the sequence of assembly of a nucleic acid, or other large molecule.

### DETAILED DESCRIPTION

### Overview

Disclosed are two new methods, SSPER and rrPCR, to introduce substitution mutations in large plasmids. The GC-rich (57-59% GC-content) Haloferax-Escherichia shuttle expression plasmids of 10 to 12 kb arc used as templates to provide examples of the types of results that can be expected when using these two methods. The SSPER and rrPCR methods readily generate the desired site-directed modifications on these large plasmids. The colonies that carried a plasmid with the desired substitution mutation arc at a rate of 50 to 100%, thus, allowing for rapid screening by DNA sequencing. The need to redesign primers for a particular mutation is eliminated, as all mutations are generated in a single strategy of primer design that allowed for flexibility in user error. The rrPCR method is also modified to generate substitution mutations at two distinct sites, as demonstrated by modification of sites spanning 69 to 996 bp distances. Overall, the SSPER and rrPCR methods are cost-effective options for generating substitution mutations in large plasmids. The methods can be performed from the first PCR reaction to the point of transforming the final PCR product into E. coli in a single day. The methods are highly accurate, require only two primers per substitution mutation, use only two enzymes (DpnI and a proofreading DNA polymerase), can be used on GC rich templates, are amenable in design, and are easily performed by entry-level researchers.

### Primer Design

The mutagenesis method disclosed include designing primers for amplifying the template and introducing the desired mutation into the template sequence. Poorly designed primers lead to reaction failure, and specific properties are needed to avoid primer-primer annealing, internal folding, or off-target annealing.

In certain embodiments, the primers pairs have selective hybridization with the nucleic acid template. Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid primer is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid primer. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, ct al., Molecular Cloning: A Laboratory Manual, Second Edition, (1989) Cold Spring Harbor, N.Y.).

In a specific embodiment, the primers are designed for site-directed mutagenesis of 1-3 nucleotides within codon sequences. Guidelines for the design of the primers are as follows. The unmodified sequence on both sides of the mutation maybe 10, 11, 12, 13, 14, 15, 16, 17, 18, 19, or 20 nt in length. In certain embodiments, the mutated bases are in the center of the mutagenic primer. The GC content of the mutagenic primers maybe at least 40%, at least 50%, at least 60%, or at least 70%. The mutagenic primers comprise a melting temperature of 70° C., 71°° C., 72°° C., 73° C., 74° C., 75° C., 76°° C., 77° C., 78° C., 79° C., or 80° C. Primer Tm values maybe calculated according to the NE BioLabs Tm calculator (https://tmcalculator.neb.com) with settings for any high-fidelity polymerase and primer concentration. The mutagenic primers start and terminate with at least one G or C and contain minimal hairpin structures. Primer hairpin structures can be examined using IDT Technologies OligoAnalyzer (https://www.idtdna.com/calc/analyzer).

### Detailed Protocols for rrPCR Method

PCR-1 reactions (100 μL) are mixed on ice in the following order:

| | | |
|---|---|---|
| 59 | μL | nuclease free water |
| 20 | μL | 5X GC buffer |
| 2 | μL | 10 mM dNTP |
| 5 | μL | 10 μM primer 1* |
| 5 | μL | 10 μM primer 2 |
| 5 | μL | DNA template (20 ng/μL or 100 ng final) |
| 3 | μL | DMSO |
| 1 | μL | Phusion DNA polymerase (2 U/μL) |
| 100 | μL | Total reaction volume |

*Throughout the entire protocol a ‘no primer’ control is included in which the volume of primers is replaced with 10 μL of nuclease free water in the PCR-1 reaction step. This control enables one to assess whether methylated plasmid DNA template is still present in the final sample that can transform E. coli in the final step 9.

PCR-1 is performed with the thermal cycler settings as listed below. A hot start setting of 98° C. is used when transferring the samples from the ice bucket into the thermal cycler.

| | | | | | | |
|---|---|---|---|---|---|---|
| 1 | cycle | 98° | C. | 4 | min | initial denaturation |
| 30 | cycles | 98° | C. | 10 | sec | denaturation |
| | | 75-80° | C.* | 30 | sec | anncaling |
| | | 72° | C. | 15 | sec** | extension |
| 1 | cycle | 72° | C. | 7 | min | final extension |

Total time 60-70 min
*A thermal gradient can be performed for annealing as needed. However, a single annealing temperature that was 3-5° C. higher than the average Tm of the primer pair sufficed as the PCR product designed were relatively short (<1 kb).
**15 to 30 sec per kb for extension.

The resulting PCR-1 mixtures are purified using a PCR cleanup kit into 80 μL (i) or 40 μL (ii) nuclease free water. The second round PCR (PCR-2) is performed in a 50 μL reaction volume by mixing on ice in the following order:

| | | |
|---|---|---|
| 37 | μL | PCR-1 mixture after PCR cleanup* |
| 10 | μL | 5X GC buffer |
| 1 | μL | 10 mM dNTP |
| 1.5 | μL | DMSO |
| 0.5 | μL | Phusion DNA polymerase (2 U/μL |
| 50 | μL | Total reaction volume |

*The original primers arc removed by the PCR cleanup. The resulting mixture includes the PCR product plus the original DNA template. A portion can be checked by DNA agarose gel electrophoresis to ensure that a PCR product is generated if needed. However, it is noted that the efficiency of generating the short (0.1 to 1 kb) PCR products of this method was high, so monitoring product formation was not necessary.

PCR-2 is performed with the thermal cycler settings as listed below. A hot start setting of 98° C. is used when transferring the samples from the ice bucket into the thermal cycler.

| 1 cycle | 98° C. | 4 | min | initial denaturation |
|---|---|---|---|---|
| 18 cycles | 98° C. | 10 | sec | denaturation |
| | 72° C. | 7 min 45 sec* | | anncaling and |
| 1 cycle | 72° C. | 10 | min | final extension |

Est total time 3.5 h**
*The temperature and time used for annealing and extension can be adjusted as needed. This adjustment was found unnecessary. For Phusion DNA polymerase, the recommended annealing time is 30 sec and the extension time is 15-30 sec per kb. The plasmids of this study were 10 to 12 kb. Thus, an extra 2 min 15 sec to 1 min 15 sec time was added to this step to ensure synthesis of the large DNA fragments.
**Each PCR reaction had the no primer control.

The resulting PCR-2 mixtures are treated with 1.5 μL DpnI (20 U/μL) for 1.5 to 3 h at 37° C. Samples are briefly chilled on ice and purified using a PCR cleanup kit into 6 μL nuclease free water. The eluted DNA is chilled on ice in a 1.5 ml microcentrifuge tube.

An appropriate number of aliquots of the E. coli TOP10 cells are thawed on ice to allow for addition of 95 μL volumes of the competent cells directly to each DNA mixture. The DNA cell mixture is gently mixed and incubated on ice for 30 min. Samples are heat treated in a 37° C. water bath for 1 min and then transferred to ice for at least 1 min. Samples are transferred to room temperature and then mixed with 1 mL LB medium using sterile technique. The samples are incubated for 1 h at 37° C. After incubation, the cells are centrifuged at room temperature for 5 min at 5000×g. The supernatant is removed. The cell pellets are resuspended in the remaining medium by vortex and then spread plated on the selective medium (LB ampicillin plates). The plates are incubated overnight at 37°° C. The number of CFUs/plate is determined for each combination of primer pair and plasmid template as well as each no primer control. Colonies (2-4) per type of site-direct mutation are picked and streaked for isolation on LB ampicillin plates.

Cells are grown overnight in 5 mL LB ampicillin medium (37° C., 200 rpm rotary shaking). Plasmid DNA is isolated from the cultures using the PureLink Quick Plasmid Miniprep Kit (Thermofisher Scientific). The DNA is eluted into 75 μL of nuclease free water.

The DNA sequence of each plasmid type is determined by Sanger DNA sequencing to identify plasmids with the site-directed mutations.

### Detailed Protocol for the SSPER Method

Single primer 1 extension reactions (50 μμL) were mixed on ice in the following order:

| | | |
|---|---|---|
| 32 | μL | nuclease free water |
| 10 | μL | 5X GC buffer |
| 1 | μL | 10 mM dNTP |
| 2.5 | μL | 10 μM primer 1 (p1)* |
| 2.5 | μL | DNA template (20 ng/μL or 50 ng final) |
| 1.5 | μL | DMSO |
| 0.5 | μL | Phusion DNA polymerase (2 U/μL) |
| 50 | μL | Total reaction volume |

*Throughout the entire protocol a no primer control was included (the volume of primers was replaced with 2.5 μL of nuclease free water).

Single primer 1 extension reactions were performed with the thermal cycler settings as listed below. A hot start setting of 98° C. was used when transferring the samples from the ice bucket into the thermal cycler.

| 1 | cycle | 98° | C. | 1 | min |
|---|---|---|---|---|---|
| 6 | cycles | 98° | C. | 10 | sec |
| | | 78° | C. | 30 sec with 0.5° C. decrease per cycle | |
| | | 72° | C. | 7 | min |
| 12 | cycles | 98° | C. | 10 | sec |
| | | 75° | C. | 30 | sec |
| | | 72° | C. | 7 | min |
| 1 | cycle | 72° | C. | 10 | min |

Total time 2.5 h

The samples from reaction 1 were digested for 2 h by addition of 1.5 μL DpnI prior to purification using a PCR cleanup kit into 40 μL nuclease free water. Single primer 2 extension reactions were performed in a 50 μL reaction volume by mixing on ice in the following order:

| | | |
|---|---|---|
| 34.5 | μL | Reaction 1 mixture after PCR cleanup* |
| 10 | μL | 5X GC buffer |
| 1 | μL | 10 mM dNTP |
| 2.5 | μL | 10 μM primer 2 (p2)** |
| 1.5 | μL | DMSO |
| 0.5 | μL | Phusion DNA polymerase (2 U/μL) |
| 50 | μL | Total reaction volume |

*The original primer 1 was removed by the PCR cleanup and the methylated template plasmid was disrupted by digestion with DpnI. The resulting mixture includes the single strand PCR product and the disrupted plasmid fragments that cannot serve as template anymore. PCR product was directly used for next stage with no need to analyze by DNA agarose gel electrophoresis.
**2.5 μL of nuclease free water was used in place of primer 2 for the no primer control.

Single primer 2 extension reactions were performed with the thermal cycler settings as listed below. A hot start setting of 98° C. was used when transferring the samples from the ice bucket into the thermal cycler.

| 1 | cycle | 98° C. | 1 | min |
|---|---|---|---|---|
| 6 | cycles | 98° C. | 10 | sec |
| | | 78° C. | 30 sec with 0.5° C. decrease per cycle | |
| | | 72° C. | 7 | min |
| 12 | cycles | 98° C. | 10 | sec |
| | | 75° C. | 30 | sec |
| | | 72° C. | 7 | min |
| 1 | cycle | 72° C. | 10 | min |

Total time 2.5 h

Samples were briefly chilled on ice and purified using a PCR cleanup kit into 6 μL nuclease free water. The eluted DNA was chilled on ice in a 1.5 ml microcentrifuge tube.

An appropriate number of aliquots of the E. coli TOP10 cells were thawed on ice to allow for addition of 95 μL volumes of the competent cells directly to each DNA mixture. The DNA cell mixture was gently mixed and incubated on ice for 30 min. Samples were heat treated in a 37° C. water bath for 1 min and then transferred to ice for at least 1 min. Samples were transferred to room temperature and then mixed with 1 mL LB medium using sterile technique. The samples were incubated for 1 h at 37° C. After incubation, the cells were centrifuged at room temperature for 5 min at 5000×g. The supernatant was removed. The cell pellets were resuspended in the remaining medium by vortex and then spread plated on the selective medium (LB ampicillin plates). The plates were incubated overnight at 37° C. The number of CFUs/plate was determined for the p1/p2 and no primer control samples.

Colonies (2 total) for the site-direct mutation (p1/p2) samples were picked and streaked for isolation on LB ampicillin plates. Cells were grown overnight in 5 mL LB ampicillin medium (37° C., 200 rpm rotary shaking). Plasmid DNA was isolated from the cultures using the PureLink Quick Plasmid Miniprep Kit (Thermofisher Scientific). The DNA was eluted into 75μL of nuclease free water. The DNA sequence of each plasmid replicate was determined by Sanger DNA sequencing to identify which of the two plasmids had the substitution mutation(s).

### EXAMPLES

### Example 1

### Methods and Materials

Strains, media and growth conditions. Strains, plasmids, and primers used are listed in Table 1. Escherichia coli strains were grown at 37° C. in Luria-Bertani (LB) medium. Media was supplemented with 1.5% agar for plates and ampicillin (Ap, 100 mg/L) as needed. Liquid cultures were grown with rotary agitation at 200 rpm. Sanger DNA sequencing of plasmids was performed by Eton Bioscience Inc. (Research Triangle Park, NC, USA) using primers p15, p16 and p10.

| Strain or | |
|---|---|
| plasmid | |
| (bp) | Description |
| E. coli | F- mcrA Δ(mrr-hsdRMS-mcrBC) Φ80lacZΔM15 ΔlacX74 |
| TOP10 | recA1 araD139 Δ(ara leu) 7697 galU galK rpsL (Strr) |
| (Invitrogen) | endA1 nupG λ- |
| pJAM503 | Apr Nvr; pBAP5010 carrying P2rrn his6-hvo_0850 (Pan1) |
| pJAM3923 | Apr Nvr; pBAP5010 carrying P2rrn his6-hvo_2995 (Fdx) |
| pJAM1208 | Apr Nvr; pBAP5010 carrying P2rrn his6-hvo_1016 |
| | (JAMM2) |
| pJAM3940 | Apr Nvr; pBAP5010 carrying P2rrn his6-hvo_1018 |
| | (RecJ3) |

TABLE 1
Lists of strains, plasmids and primers used in this studyª.
ªAp^{r}, ampicillin resistance; Nv^{r}, novobiocin resistance; Str^{r}, streptomycin resistance.
P2_{rrnA}, rRNA promoter used for gene expression. Open reading frames targeted for mutagenesis:
hvo_0850 (Pan1, UniProt D4GUJ7); hvo_2995 (Fdx, D4GY89); hvo_1016 (JAMM2), and hvo_1018
(RecJ3, D4GVJ5) were fused to an N-terminal histidine tag (his6-). Primers p1 to p15 were
used for SDM with the altered nucleotides written in lowercase characters. p16 and p17
primers were used for DNA sequencing. Primer and hairpin Tm's were calculated as described
in methods.

| | | SEQ | | | | |
|---|---|---|---|---|---|---|
| | | ID | | | Primer | Hairpin |
| Primer | | NO | Sequence (5′-3′) | Length | Tm | Tm |
| p1 | JAMM2Y117 > | 1 | CGGCTCGCCGTAgGGCCCCGACGAC | 25 nt | 78° C. | 51.8° C. |
| | TAGf | | | | | |
| p2 | JAMM2Y117 > | 2 | GTCGTCGGGGCCcTACGGCGAGCCG | 25 nt | 78° C. | 58.1° C. |
| | TAGr | | | | | |
| p3 | FdxF1 | 3 | GTGAGATGGAAGTGAACCAGGGCGAGTAC | 29 nt | 70° C. | 35.4° C. |
| p4 | FdxF2 | 4 | CCATCGTGAAGGAGGGCGAAATCGACATG | 29 nt | 70° C. | 49.4° C. |
| p5 | Fdx K119Q | 5 | GTAGTCGAGGTGCTgCGCGTTGTAGAC | 27 nt | 70° C. | 36.9° C. |
| p6 | Fdx K119R | 6 | GTAGTCGAGGTGCcgCGCGTTGTAGAC | 27 nt | 72°°C. | 26.4° C. |
| p7 | Fdx K119TAG | 7 | GTAGTCGAGGTGCTaCGCGTTGTAGACGATC | 31 nt | 70° C. | 55.2° C. |
| p8 | Fdx K97Q | 8 | GAAGTCAACGAGCAGAACGTCCGCCTC | 27 nt | 70° C. | 40.9° C. |
| p9 | Pan1-K214 > | 9 | CGCCTCGTTCATCtagATGGCCGGCTCCG | 29 nt | 75° C. | 41.7° C. |
| | TAGf | | | | | |
| p10 | Pan1-int1 | 10 | CGATGGCGTCTATCTCGTCGATG | 23 nt | 64° C. | 34.6° C. |
| p11 | Pan1-F284 > | 11 | GCCGCGCTCGTCctaGCCGTCCATCTC | 27 nt | 75° C. | 35.2° C. |
| | TAGr | | | | | |
| p12 | RecJ3D265Af | 12 | CCGACGGCGcCGGCATGTGCG | 21 nt | 75° C. | 49° C. |
| p13 | RecJ3R567Ar | 13 | ACGCCGTCGGAGgcGAGGACGGCGAAG | 27 nt | 78° C. | 80.7° C. |
| p14 | RecJ3H596Ar | 14 | CCGACGACGAGGgcGCCGCCGCCGGAG | 27 nt | 82° C. | 57.8° C. |
| p15 | Pan1-F214 > | 15 | CGGAGCCGGCCATctaGATGAACGAGGCG | 29 nt | 80° C. | 42.2° C. |
| | TAGr | | | | | |
| p16 | HvPrrnF | 16 | CGATGCCCTTAAGTACAACAGGGT | 24 nt | 64° C. | 48.5° C. |
| p17 | T7TerR | 17 | AACCCCTCAAGACCCGTTTAGAG | 23 nt | 64° C. | 25.5° C. |

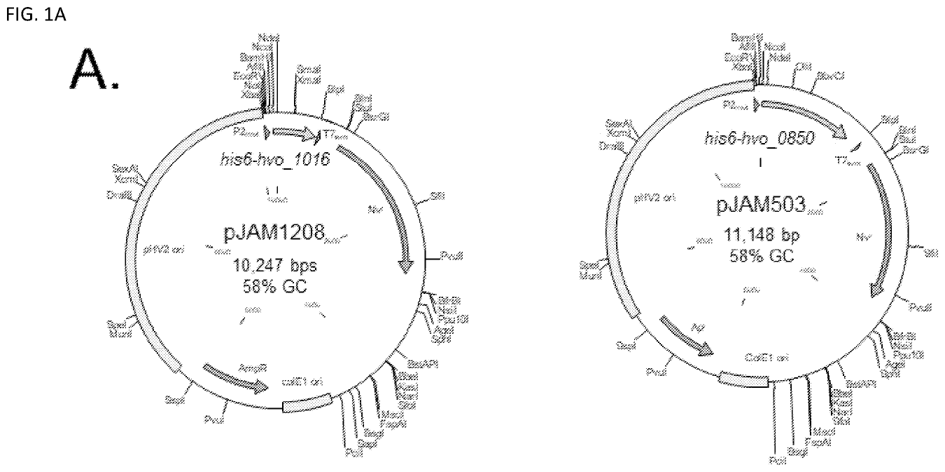

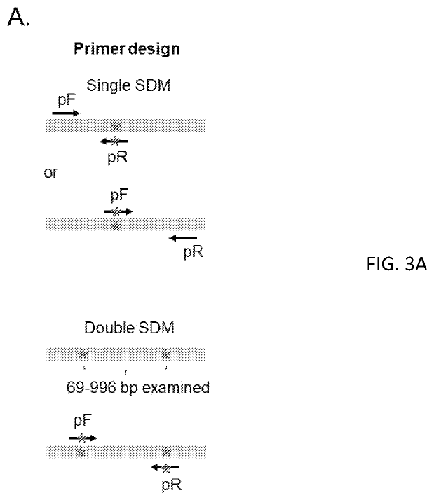

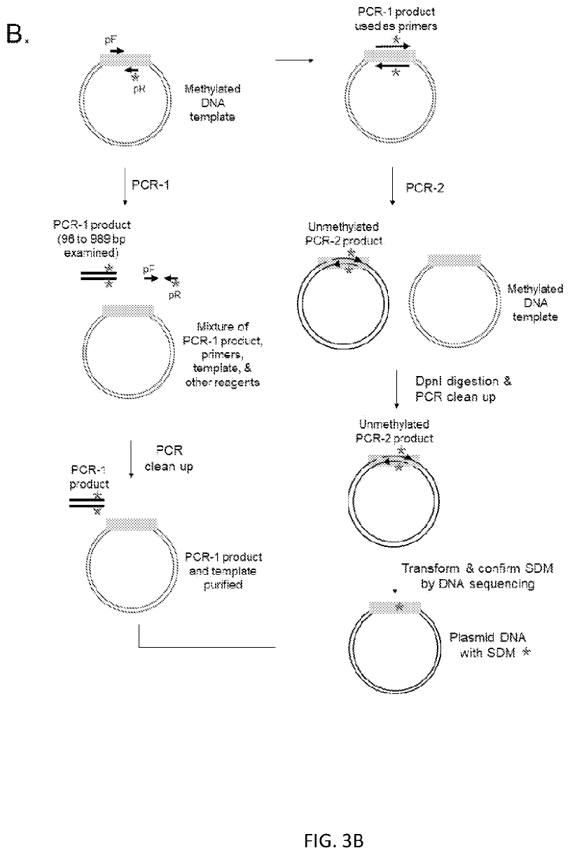

Primer design. Primers (SED ID List; Table 1) were designed for site-directed mutagenesis of 1-3 nucleotides within codon sequences (FIGS. 1A, 3A). Guidelines for the design of the primers were: 10-20 nt of unmodified sequence on both sides of the mutation; mutated bases in the center; GC content of 40-70%; Tm of 70-80° C.; start and terminate with at least one G or C; and minimal hairpin structures. To test the robustness of the rrPCR method, primers were also tested with: i) Tm outside the 70-80° C. range (p14 and p10), ii) extensive hairpin structures (p13), and iii) sequence starting with an A (p13). Primer Tm values were calculated according to the NE BioLabs Tm calculator (https://tmcalculator.neb.com) with settings for Phusion high-fidelity DNA polymerase in GC buffer and 500 nM primer concentration. Primer hairpin structures were examined using IDT Technologies OligoAnalyzer (https://www.idtdna.com/calc/analyzer) with settings at Oligo Conc 0.5 μM, Na+ Conc 50 mM, Mg++ Conc 1.5 mM and dNTPs Conc 0.2 mM.

Plasmid DNA template. Haloferax-Escherichia shuttle expression plasmids pJAM503, pJAM3923, pJAM1208 and pJAM3940 (Table 1) were used as the methylated DNA templates in the first step of the SSPER or rrPCR methods, as indicated. The plasmids were propagated in E. coli TOP10 (dam+dcm+) and then isolated from 5 mL overnight cultures using the PureLink Quick Plasmid Miniprep Kit (Thermofisher Scientific). The DNA was eluted into 75 μL of nuclease free water and stored at −20° C. until use. The concentration of the plasmid DNA was determined using a microvolume spectrophotometer (Take3 micro-volume plate with Gen5 software, BioTek Instruments) and was at a typical yield of 80-100 ng/μL. The plasmid DNA was diluted in nuclease free water to 20 ng/μL for use as template in either the PCR-1 or single primer extension reactions (the first step of rrPCR and SSPER, respectively). Preparation of competent cells for transformation of plasmid DNA. E. coli TOP10competent cells were generated for transformation of plasmid DNA as follows. CCMB80 buffer was prepared (1 L): 10 mM potassium acetate pH 7.0 (10 ml of 1M stock), 80 mM CaCl₂·2H₂O (11.8 g), 20 mM MnCl₂·4H₂O (4.0 g), 10 mM MgCl₂·6H₂O (2.0 g), 10% glycerol (100 ml), adjusted to pH 6.4 with 0.1N HCl as needed. CCMB80 buffer was sterile filtered and stored at 4° C. until use. TOP10 cells were streaked for isolation onto SOB agar plates and grown overnight at room temperature. Isolated colonies were picked into 10 mL of SOB medium (125 mL

Erlenmeyer flask) and incubated overnight at room temperature (200 rpm, orbital shaking). Once grown, the culture was mixed with 6 mL 40% v/v glycerol, distributed in 1 mL aliquots into cryotubes and stored at −80°° C. One vial of the seed stock (1 mL) was thawed and used to inoculate 250 ml of SOB medium (500 mL Erlenmeyer flask). Cells were grown to an OD600 nm of 0.2 to 0.3 at room temperature (200 rpm, orbital shaking). Cultures were incubated on ice (10 min) and harvested by centrifugation in 50 mL sterile conical tubes (1378×g, 4° C. for 10 min). The supernatant was completely removed, and the cells were gently resuspended in 80 ml CCMB80 buffer (ice cold) using a 10 mL pipette. The mixture was chilled on ice (20 min) and, after this incubation, was similarly harvested and resuspended into 10 ml CCMB80 buffer (ice cold). Cells (100 μl) were aliquoted into 1.8 mL microcentrifuge tubes and stored at −80° C.

An appropriate number of aliquots of the E. coli TOP10 cells were thawed on ice to allow for addition of 95 μL volumes of the competent cells directly to each DNA mixture. The DNA cell mixture was gently mixed and incubated on ice for 30 min. Samples were heat treated in a 37° C. water bath for 1 min and then transferred to ice for at least 1 min. Samples were transferred to room temperature and then mixed with 1 mL LB medium using sterile technique. The samples were incubated for 1 h at 37° C. After incubation, the cells were centrifuged at room temperature for 5 min at 5000×g. The supernatant was removed. The cell pellets were resuspended in the remaining medium by vortex and then spread plated on the selective medium (LB ampicillin plates). The plates were incubated overnight at 37° C.

The number of CFUs/plate was determined for the p1/p2 and no primer control samples. Colonies (2 total) for the site-direct mutation (p1/p2) samples were picked and streaked for isolation on LB ampicillin plates. Cells were grown overnight in 5 mL LB ampicillin medium (37° C., 200 rpm rotary shaking). Plasmid DNA was isolated from the cultures using the PureLink Quick Plasmid Miniprep Kit (Thermofisher Scientific). The DNA was eluted into 75 μL of nuclease free water. The DNA sequence of each plasmid replicate was determined by Sanger DNA sequencing to identify which of the two plasmids had the substitution mutation(s).

### Example 2

### Standard PCR and its Limitation in Generating Site-Directed Mutations in Large Plasmids

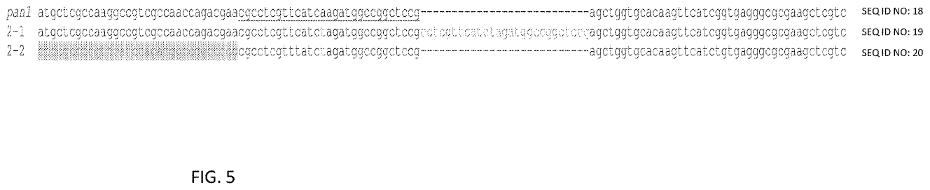

In the initial attempt to generate substitution mutations in large plasmids (>10 kb), the conventional primer design was used, where the two primers were complementary with the substitution mutation positioned in the center of each primer. Primer pairs p1/p2 and p9/p15 were designed to be used with templates pJAM1208 and pJAM503, respectively (Table 1, FIG. 1A-B). The plasmid DNA templates were propagated in E. coli TOP10 and purified using a conventional miniprep kit. As this strain is dam+dem+, the plasmids were methylated by the E. coli strain at the common Dam and Dem sites making the DNA template and any hemimethylated DNA derivates susceptible to DpnI digestion at later stages in the protocol [12]. Standard PCRs was performed by mixing both the mutagenesis primers and the plasmid template in the same reaction. The PCR products were subsequently digested with DpnI to remove the template and then transformed into E. coli TOP10. By this conventional approach, the number of colonies obtained on the selection plates was reproducibly negligible if any for the test samples when compared to the no primer control (FIG. 1B). The few plasmids that were analyzed from the ‘positive’ colonies by DNA sequencing either corresponded to the original template or had unusual rearrangements that suggested primer dimers formed, thus, complicating the results (FIG. 5).

### Example 3

### Sequential Single Primer Extension Reaction (SSPER) Method to Generate Substitution Mutations

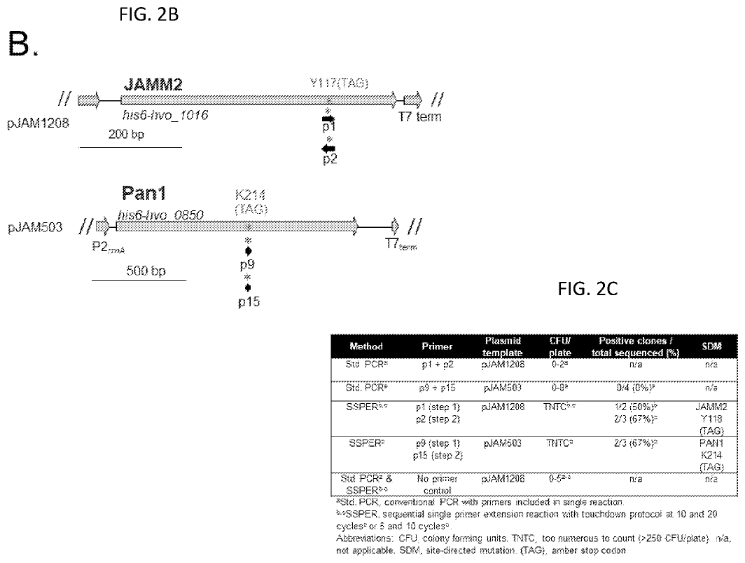

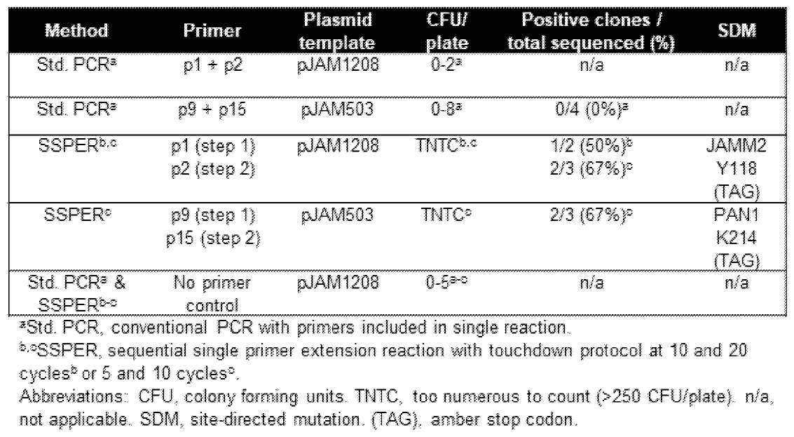

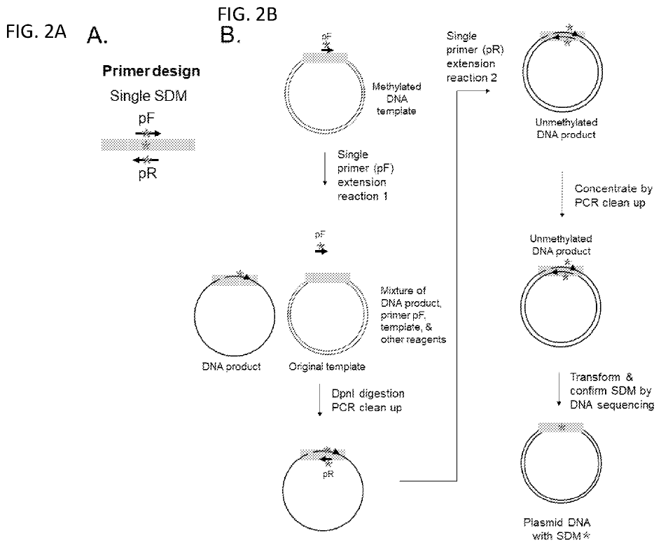

The SSPER method was developed: i) to minimize the formation of primer cross dimers or other artifacts that occur due to the annealing of short complementary ssDNA primers, and ii) to avoid the rearrangements and other problems that may arise when annealing two large strands of DNA in vitro [17]. The SSPER method used the conventional primer design, where the substitution mutation(s) were positioned in the center of each primer, and the primers were fully complementary (FIG. 2A). Furthermore, the plasmid DNA template was prepared in a methylated state from an E. coli dam+dcm+ strain (FIG. 2B). However, the feature that distinguished SSPER from other methods was the use of sequential primer extension reactions, with the first primer removed from the sample after the first primer extension reaction was completed and the second primer added to this purified sample for the final round of primer extension to generate the desired product (FIG. 2B). By contrast, the traditional approach includes both ssDNA primers in the same PCR, while SPRINP separates the ssDNA primers into two parallel primer extension reactions but relies upon annealing the two products in vitro after the primer extension reactions are separately performed [17].

In the SSPER method, as outlined in FIG. 2, primers (p1 and p2) are designed in the traditional manner, as complementary with the substitution mutation(s) positioned in the center. One primer (e.g., p1) is selected for use in the first single primer extension reaction and the methylated plasmid DNA is used as template. In the extension reaction, the annealing temperature is gradually reduced using a touchdown program to maximize the specificity of annealing the 25 oligonucleotide ssDNA primer to its target. After extension, the first primer (p1) is removed from the sample by PCR cleanup. The mixture of the original DNA template and the newly synthesized ssDNA is then used in a similar single primer extension reaction with the second primer (e.g., p2). The desired target for this second extension reaction is the large synthetic ssDNA fragment. The original methylated DNA template and any hemimethylated derivatives are removed by DpnI digestion. At the final stage, the SSPER-generated DNA product with the site-directed mutation is transformed into E. coli.

To test the SSPER method, primers p1 and p2 were used sequentially with the E. coli methylated plasmid pJAM1208 as the template and examined use of DpnI digestion before or after the second extension reaction (FIG. 1). For all SSPER approaches examined, the background was low at only 0 to 5 CFUs/plate detected when the DNA products of the no primer control were transformed into E. coli (FIG. 1B). Furthermore, all SSPER approaches gencrated >250 CFUs/plate when the DNA products generated by site-directed mutagenesis were transformed into E. coli (FIG. 1B).

One complication with the initial SSPER protocol was that each single primer extension reaction lasted 4.5 hours. As two sequential primer extension reactions were used to generate the final product, the total time on the thermocycler was 9 hours which created a bottleneck in the lab. A long extension time appeared important in the reaction series to ensure the >10 kb product was fully synthesized. In addition, a ‘touchdown’ approach was used to enhance primer annealing to the large DNA template consisting of a decrease in annealing temperature of 0.5° C. per cycle for 10 cycles followed by 20 cycles at the target annealing temperature. As the primers were only 25 nucleotides and the template was >10 kb, the touchdown approach was included to enhance annealing specificity. However, this approach prolonged the single primer extension reaction time.

### Example 4

### Optimization of SSPER

To remedy the long incubation times on the thermocycler, it was examined whether the number of cycles could be reduced in the SSPER protocol. This approach was tested using p1/p2 and p9/p15 primer pairs with pJAM1208 and pJAM503 as templates, respectively (FIG. 1AB). The long extension time was still used to ensure the >10 kb products were synthesized. However, the number of cycles was decreased from 10 to 6 cycles during the 0.5° C. per cycle decrease in annealing temperature and from 20 to 12 cycles once the annealing temperature target was reached. Overall, the total thermocyler time for the SSPER method was reduced from 9 hours (2×4.5 hours) to 5 hours (2×2.5 hours), while the number of colonies observed for the site-directed mutagenesis samples remained high (>250 CFUs/plate) compared to limited number of colonics detected in the no primer control. Further analysis by DNA sequencing, revealed that the substitution mutations were generated using the p1/p2 and p9/p15 primer pairs with the pJAM1208 and pJAM503 templates at a rate of 67% (2 out of 3 clones), respectively. Thus, the SSPER method with the 4-hour reduction in overall reaction time appears efficient at generating site-directed mutations in large plasmids.

To achieve the highest efficiency with SSPER method, the DpnI digestion step was moved to between the two single primer PCRs, thus in the second single primer PCR only the newly synthesized ssDNA serve as template, allowing the theoretical efficiency of getting mutated molecules to approach 100%. The sequencing result with this method proved four out of four sequenced colons showed correct substitution mutations.

### Example 5

### Reduce Recycle PCR (rrPCR) Method to Generate Substitution Mutations

Next, the substitution mutagenesis was approached from a slightly different angle. Like SSPER, the approach still incorporated the removal of short ssDNA primers at an intermediate stage in the method, but the distinction from SSPER was that the primers were not complementary and that the second reaction in the series used the PCR products as primers. The lack of complementarity in the initial primer design allowed substitution mutations to be incorporated at one or two distinct sites on the target sequence and the same ‘anchor’ primer to be used for multiple substitution mutations. This approach is the reduce recycle PCR (rrPCR) method, to highlight how the number of synthetic primers is reduced and the template is recycled. The concept behind rrPCR is to design the primers as forward and reverse primers (pF and pR) in a traditional PCR style; however, a substitution mutation(s) is included in the center of one or both primers (FIG. 3A). A traditional PCR is performed using methylated plasmid DNA as template (FIG. 3B). The innovation in the approach is that after the first PCR (PCR-1), the ssDNA primers are removed by PCR clean up and the resulting mixture is used for a second round of PCR (PCR-2) with no external primers or template added. In the PCR-2 stage, the dsDNA product of PCR-1 serves as the primer pair and the recycled methylated plasmid

DNA serves again as the template. After PCR-2, the template and any other hemimethylated/methylated DNA fragments are removed by DpnI digestion prior to transformation of E. coli. The advantages of the rrPCR approach over SSPER are that substitution mutations can be incorporated in two distinct sites in parallel, the number of primers is reduced, and a thermocycler with touchdown options is not needed.

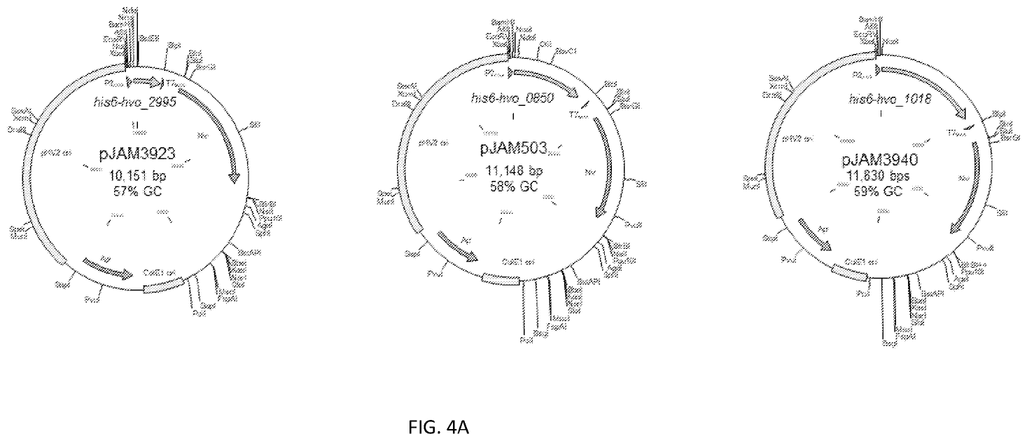

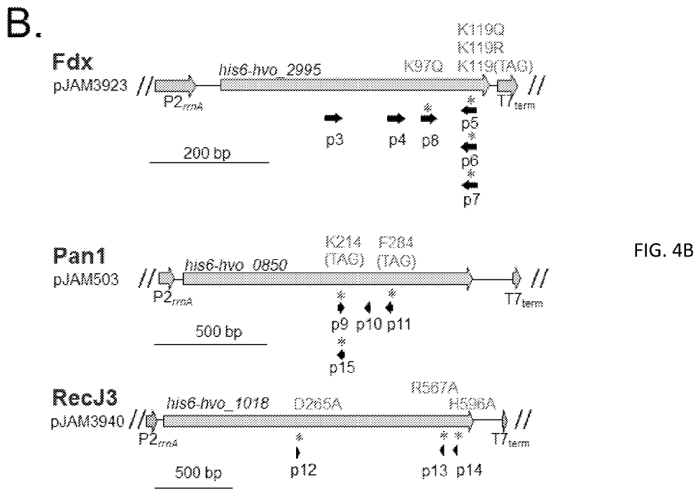

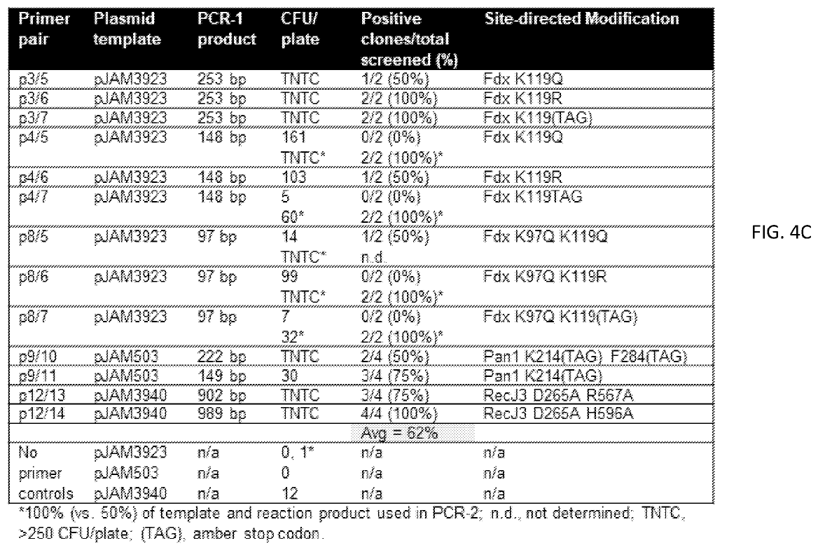

To demonstrate the efficacy of the rrPCR method, substitution mutations were introduced at nine different sites in single and double combinations (separated by 69 to 996 bp) on three different plasmid templates (pJAM3923, pJAM503 and pJAM3940) ranging in size from 10.2 to 11.8 kb and GC content from 57 to 59% (FIG. 4A). Most primers were designed within the guidelines of: 10-20 nt of unmodified sequence on both sides of the mutation; mutated bases in the center; GC content of 40-70%; Tm of 70-80° C.; start and terminate with at least one G or C; and minimal hairpin structures. However, to test the robustness of the rrPCR method, a subset of the primers was designed to have suboptimal features including: i) a Tm outside of the 70-80° C. range (p10 and 14), ii) extensive hairpin structures (p13), and iii) sequence starting with an A (p13). These suboptimal primers (p10, 13, and 14) were still successful in generating the desired substitution mutations at a rate of 50-75% of the clones screened. Of the 13 different primer pair combinations examined, all were productive in generating the desired substitution mutation(s) with high efficiency and yield (FIG. 4B). In the case of primer pair combinations p4/5, p4/7,p8/6 and p8/7, transferring 100% (and not 50%) of the PCR-1 mixture to the PCR-2 stage was found to generate the desired product. The other primer pair combinations were successful when 50% of the PCR-1 mixture was used for the next round of PCR (PCR-2). Each substitution type could be identified through DNA sequencing of only 2-4 clones without the need to pre-screen clones using protocols such as the introduction of silent restriction modification sites [18]. When using rrPCR, isolation of plasmids with DNA artifacts or the original DNA sequence was minimal. For comparison, significant rearrangements occurred in the few plasmid DNA products isolated when using the standard PCR with complementary primers p9 and p15 and plasmid pJAM503 as the template, thus, preventing generation of the Pan1 K214 to amber stop codon (TAG) variant by the conventional method (FIG. 5).

The high efficiency of the rrPCR method compared to the methods that incorporate short complementary primers in the same PCR may be explained by the following points. First, the amplified “long primer” has a much higher specificity than the short oligonucleotides toward annealing to the plasmid template. Second, the dynamics of the annealing of the amplified “long primer” (PCR-1 product) to the template is different than the short primers. This difference is due to the frequency of forming cross dimers between the primer pairs, which is much lower between the amplified complementary “long primers” than that of the short primers. Thus, the annealing of the “long primers” to the template can compete with forming cross dimers to a higher extent than the short primers. By contrast, the efficiency of forming cross dimers for the short complementary primers is so high that there are limited effective free primers working in the PCR reaction to anneal to the template DNA.

### REFERENCES

1. Zawaira A, Pooran A, Barichievy S, Chopera D: A discussion of molecular biology methods for protein engineering. Mol Biotechnol 2012, 51 (1): 67-102.
2. Ling M M, Robinson B H: Approaches to DNA mutagenesis: an overview. Anal Biochem 1997, 254 (2): 157-178.
3. Watson J F, García-Nafría J: DNA assembly using common laboratory bacteria: A re-emerging tool to simplify molecular cloning. J Biol Chem 2019, 294 (42): 15271-15281.
4. Shilling P J, Mirzadeh K, Cumming A J, Widesheim M, Kock Z, Daley D O: Improved designs for pET expression plasmids increase protein production yield in Escherichia coli. Commun Biol 2020, 3 (1): 214.
5. Jia H, Couto-Rodriguez R L, Gal D, Mondragon P, Wassel P C, Yu D, Maupin-Furlow J A: Expression and tandem affinity purification of 20S proteasomes and other multisubunit complexes in Haloferax volcanii. Methods Enzymol 2021, 659:315-326.
6. Kasirajan L, Adams Z, Couto-Rodriguez R L, Gal D, Jia H, Mondragon P, Wassel P C, Yu D, Uthandi S, Maupin-Furlow J A: High-level synthesis and secretion of laccase, a metalloenzyme biocatalyst, by the halophilic archaeon Haloferax volcanii. Methods Enzymol 2021, 659:297-313.
7. Aiyar A, Xiang Y, Leis J: Site-directed mutagenesis using overlap extension PCR. Methods Mol Biol 1996, 57:177-191.
8. Ho S N, Hunt H D, Horton R M, Pullen J K, Pease L R: Site-directed mutagenesis by overlap extension using the polymerase chain reaction. Gene 1989, 77 (1): 51-59.
9. Barettino D, Feigenbutz M, Valcárcel R, Stunnenberg H G: Improved method for PCR-mediated site-directed mutagenesis. Nucleic Acids Res 1994, 22 (3): 541-542.
10. Giebel L B, Spritz R A: Site-directed mutagenesis using a double-stranded DNA fragment as a PCR primer. Nucleic Acids Res 1990, 18 (16): 4947.
11. Tseng W C, Lin J W, Wei T Y, Fang T Y: A novel megaprimed and ligase-free, PCR-based, site-directed mutagenesis method. Anal Biochem 2008, 375 (2): 376-378.
12. Buryanov Y, Shevchuk T: The use of prokaryotic DNA methyltransferases as experimental and analytical tools in modern biology. Anal Biochem 2005, 338 (1): 1-11.
13. Costa G L, Bauer J C, McGowan B, Angert M, Weiner M P: Site-directed mutagenesis using a rapid PCR-based method. Methods Mol Biol 1996, 57:239-248.
14. Zheng L, Baumann U, Reymond J L: An efficient one-step site-directed and site-saturation mutagenesis protocol. Nucleic Acids Res 2004, 32 (14): e115.
15. Zhang B Z, Zhang X, An X P, Ran D L, Zhou Y S, Lu J, Tong Y G: An easy-to-use site-directed mutagenesis method with a designed restriction site for convenient and reliable mutant screening. J Zhejiang Univ Sci B 2009, 10 (6): 479-482.
16. Zhang K, Yin X, Shi K, Zhang S, Wang J, Zhao S, Deng H, Zhang C, Wu Z, Li Y et al: A high-efficiency method for site-directed mutagenesis of large plasmids based on large DNA fragment amplification and recombinational ligation. Sci Rep 2021, 11 (1): 10454.
17. Edelheit O, Hanukoglu A, Hanukoglu I: Simple and efficient site-directed mutagenesis using two single-primer reactions in parallel to generate mutants for protein structure-function studies. BMC Biotechnol 2009, 9:61.
18. Karnik A, Karnik R, Grefen C: SDM-Assist software to design site-directed mutagenesis primers introducing “silent” restriction sites. BMC Bioinformatics 2013, 14:105.

What is claimed is:

1. A method for site-directed mutagenesis comprising:
a. Combining a reaction mixture, said reaction mixture comprising a nucleic acid template, a first mutagenic primer, a second primer, said second primer optionally being a mutagenic primer, and a high-fidelity polymerase;
b. Annealing the first mutagenic primer and second primer to the nucleic acid template;
c. Amplifying the nucleic acid template to produce a first nucleic acid product, wherein the first mutagenic primer introduces a first mutation to the first nucleic acid product and optionally the second primer, if mutagenic, introduces a second mutation to the first nucleic acid product;
d. removing the first mutagenic primer and second primer from the reaction mixture;
e. Annealing the first nucleic acid product containing the mutation to the nucleic acid template;
f. Amplifying the nucleic acid template to produce a second nucleic acid product, wherein the first nucleic acid product introduces the mutation to the second nucleic acid product; and
g. removing the nucleic acid template.

2. The method of claim 1, wherein the first nucleic acid product comprises a length of 96 nt to 989 nt.

3. The method of claim 1, wherein the nucleic acid template comprises a length greater than 10 kb.

4. The method of claim 1, wherein the first mutagenic primer and/or the second primer comprise a length of 10 nt to 20 nt.

5. The method of claim 1, wherein the first mutagenic primer and the second primer comprise a GC content of 40% to 70%.

6. The method of claim 1, wherein the first mutagenic primer and the second primer comprise a melting temperature of 70°° C. to 80° C.

7. The method of claim 1, wherein the first and second mutations are introduced to two distinct sites in the nucleic acid product.

8. The method of claim 7, wherein the first and second mutations are separated by 69 bp to 996 bp on the nucleic acid product.

9. The method of claim 1, wherein the first mutagenic primer and the second primer are removed by PCR cleanup.

10. The method of claim 1, wherein annealing and amplifying are performed in a thermocycler.

11. The method of claim 1, wherein the mutations are a substitution mutation.

12. The method of claim 1, wherein the nucleic acid template is removed by a methylation sensitive restriction enzyme.

13. The method of claim 1, wherein the method comprises a mutation rate of 50% to 100%.

14. A method for site-directed mutagenesis comprising:
a. Combining a reaction mixture, said reaction mixture comprising a nucleic acid template, a first mutagenic primer, and a high-fidelity polymerase;
b. Annealing the first mutagenic primer to the nucleic acid template;
c. Amplifying the nucleic acid template to produce a single-strand nucleic acid product, wherein the first mutagenic primer introduces a mutation to the single-strand nucleic acid product;
d. Removing the first mutagenic primer and the nucleic acid template from the reaction mixture;
e. Adding a second mutagenic primer to the reaction mixture
f. Annealing the second mutagenic primer to the single-strand nucleic acid product;
g. Amplifying the single strand nucleic acid product to produce a double strand nucleic acid product containing the mutation; and
h. Optionally, removing any derivatives.

15. The method of claim 14, wherein the nucleic acid template comprises a length greater than 10 kb.

16. The method of claim 14, wherein the first mutagenic primer comprises a sequence that is complementary to the second mutagenic primer.

17. The method of claim 14, wherein the first mutagenic primer is removed by PCR cleanup.

18. The method of claim 14, wherein annealing and amplifying are performed in a thermocycler.

19. The method of claim 14, wherein the mutation is a substitution mutation.

20. The method of claim 14, wherein the nucleic acid template and the derivatives arc removed by a methylation sensitive restriction enzyme.

21. The method of claim 14, wherein the method comprises a mutation rate of at least 67%.

22. The method of any of claims 1-13, wherein the nucleic acid template is methylated.

23. The method of claim 23, wherein the second nucleic acid product is unmethylated.

24. The method of any of claim 1-13, 22 or 23, wherein the resulting second gene product is introduced into a bacterial cell.

25. The method of claim 24, wherein the bacterial cell is an E. coli cell.

26. The method of claim 24 or 25, wherein the second gene product is introduced as a plasmid.

27. The method of any of claims 1-14, wherein the nucleic acid template is methylated.

28. The method of claim 27, wherein the double stranded nucleic acid product is unmethylated.

29. The method of any of claim 14-21, 27 or 28, wherein the double stranded nucleic acid product is introduced into a bacterial cell.

30. The method of claim 29, wherein the bacterial cell is an E. coli cell.

31. The method of claim 29 or 30, wherein the double stranded nucleic acid product is introduced as a plasmid.
